(19) United States
(12) Patent Application Publication
(10) Pub. No.: US 2017/0252582 A1
(43) Pub. Date: Sep. 7, 2017

(54) ULTRASOUND EMISSION DEVICE

(71) Applicant: Seiko Epson Corporation, Tokyo (JP)

(72) Inventors: Robina ATSUCHI, Fujimi (JP); Daisuke NAKANISHI, Matsumoto (JP)

(21) Appl. No.: 15/440,499

(22) Filed: Feb. 23, 2017

(30) Foreign Application Priority Data
Mar. 1, 2016 (JP) 2016-038647

(51) Int. Cl. A61N 7/00

(52) U.S. Cl. CPC A61N 7/00

(57) ABSTRACT

An ultrasound emission device includes an ultrasound emission unit that emits an ultrasound, a drive unit that drives the ultrasound emission unit, and a support unit that supports the ultrasound emission unit and the drive unit so as to be installed in a human body. The ultrasound emission unit has a shape to be installed in an external auditory canal of the human body. The support unit has a shape to be installed in an auditory capsule of the human body. The ultrasound emission unit has a through-hole installed in order to transmit a sound to an eardrum of the human body.

### BACKGROUND

1. Technical Field

The present invention relates to an ultrasound emission device.

2. Related Art

Researches have been actively carried out in order to study a change in brain activity by emitting an ultrasound having moderate sound pressure to a brain. Then, the researches have confirmed a phenomenon that the brain activity is activated by emitting the ultrasound to the brain. A report has been published that an examinee who comes into contact with the emitted ultrasound feels relaxed and motivated. Specifically, the ultrasound which vibrates approximately 10 million times per second is emitted. In this manner, a microtubule present in a brain site associated with feelings can be resonated with substantially the same frequency. The microtubule has a tubular structure with a diameter of approximately 25 nm, which is found in a cell. Alternatively, studies have been progressively developed in which the ultrasound is emitted to the brain so as to recover a memory or a brain function itself lost from the brain having an Alzheimer's disease.

JP-T-2013-509958 discloses a device which emits the ultrasound to the brain. According to this device, an ultrasound transducer is installed in a helmet, and the ultrasound is emitted from the ultrasound transducer toward the brain. A phased transducer array is used in order to focus the ultrasound on a specific location after the ultrasound passes through a skull.

The device disclosed in JP-T-2013-509958 is a helmet-shaped device. An operator wears the device on a head. The skull is present between the ultrasound transducer and the brain. The ultrasound is less likely to be transmitted through the skull. Accordingly, it is necessary to emit the ultrasound having great sound pressure from the ultrasound transducer. Therefore, since the ultrasound transducer is likely to consume power, the device needs a large capacity battery. As a result, a heavy battery is installed in the device.

In this case, since the battery is heavy, a heavy load is applied to the head. A head shape of a human body varies depending on persons. Thus, it is difficult to fix the device. Since the operator moves in a state where the heavy device is fixed to the head, the operator needs to move slowly. Accordingly, the operator wearing the device is less likely to spend a normal life. Therefore, an ultrasound emission device has been desired which can transmit the ultrasound to the brain even when a person moves during a daily life.

### SUMMARY

An advantage of some aspects of the invention is to solve at least a part of the problems described above, and the invention can be implemented as the following forms or application examples.

### APPLICATION EXAMPLE 1

An ultrasound emission device according to this application example includes: an ultrasound emission unit that emits an ultrasound; a drive unit that drives the ultrasound emission unit; and a support unit that supports the ultrasound emission unit and the drive unit so as to be installed in a human body. The ultrasound emission unit has a shape to be installed in an external auditory canal of the human body. The support unit has a shape to be installed in an auditory capsule of the human body.

According to this application example, the ultrasound emission device includes the ultrasound emission unit, the support unit, and the drive unit. The ultrasound emission unit emits the ultrasound, and the support unit supports the ultrasound emission unit. The drive unit drives the ultrasound emission unit. In this manner, the ultrasound emission device can transmit the ultrasound to a brain from the external auditory canal of the human body. The human body receives the ultrasound from the external auditory canal, thereby enabling the brain to be activated and awakened. The support unit installs the ultrasound emission unit and the drive unit in the human body. Therefore, even when the human body moves, the ultrasound can be emitted toward the human body.

The ultrasound emission unit has the shape to be installed in the external auditory canal of the human body. The support unit has the shape to be installed in the auditory capsule of the human body. Therefore, the ultrasound can be emitted to the external auditory canal by installing the ultrasound emission device in the ear of the human body. The brain is surrounded with the skull, and the ultrasound is less likely to be transmitted through the skull. On the other hand, since a hole is present in the skull in a site having the external auditory canal, even low sound pressure enables the ultrasound to be propagated to the brain. Therefore, even if the ultrasound emission device employs a small capacity battery which is light in weight, the ultrasound can be emitted to the brain for a long period of time. As a result, even when the human body moves during a daily life, the ultrasound can be transmitted to the brain.

### APPLICATION EXAMPLE 2

In the ultrasound emission device according to the application example, the ultrasound emission unit may have a hole installed in order to transmit a sound to an eardrum of the human body.

According to this application example, the hole is installed in the ultrasound emission unit. The sound can be transmitted to the eardrum of the human body through the hole. Therefore, even while the ultrasound is emitted to the brain, the human body can hear the sound propagated in the air around the human body.

### APPLICATION EXAMPLE 3

The ultrasound emission device according to the application example may further include a speaker that emits a voice.

According to this application example, the ultrasound emission device includes the speaker that emits the voice. Therefore, a stimulus using the ultrasound and a stimulus using the voice can be simultaneously given to the human body.

### APPLICATION EXAMPLE 4

An ultrasound emission device according to this application example includes: an ultrasound emission unit that emits an ultrasound; a drive unit that drives the ultrasound emission unit; and a support unit that supports the ultrasound emission unit and the drive unit so as to be installed in a human body. The support unit has a shape to be installed on a neck of the human body. The ultrasound emission unit emits the ultrasound toward the neck of the human body.

According to this application example, the ultrasound emission unit is installed on the neck of the human body by using the support unit. The ultrasound emission unit emits the ultrasound toward the neck of the human body. Therefore, the ultrasound can be emitted toward the neck by installing the ultrasound emission device on the neck of the human body. As a result, the ultrasound emission device can give a stimulus of the ultrasound to the brain by way of an artery or a vein of the neck.

The brain is surrounded with the skull, and the ultrasound is less likely to be transmitted through the skull. On the other hand, since the neck has no skull, even low sound pressure enables the ultrasound to be propagated to the artery and the vein. A stimulus of the ultrasound can be propagated to the brain via the artery and the vein. Therefore, even if the ultrasound emission device employs a small capacity battery which is light in weight, the ultrasound can be emitted to the artery and the vein for a long period of time. As a result, even when the human body moves during a daily life, the stimulus can be transmitted to the brain by emitting the ultrasound.

### APPLICATION EXAMPLE 5

In the ultrasound emission device according to the application example, the ultrasound emission unit may have an emission direction adjustment unit that changes a direction of a surface from which the ultrasound is emitted.

According to this application example, the ultrasound emission unit has the emission direction adjustment unit that changes the direction of the surface from which the ultrasound is emitted. Therefore, the surface from which the ultrasound is emitted can match a surface of the neck. As a result, the ultrasound can be efficiently transmitted to the artery and the vein.

### APPLICATION EXAMPLE 6

In the ultrasound emission device according to the application example, the support unit may have a support adjustment unit that is deformed so as to match the neck of the human body.

According to this application example, the support unit has the support adjustment unit that is deformed so as to match the neck of the human body. In this manner, a configuration can be adopted in which even if the human body moves, the ultrasound emission device is less likely to be moved from the neck. Therefore, even if a person moves, the ultrasound emission device can stably emit the ultrasound to the artery and the vein.

### BRIEF DESCRIPTION OF THE DRAWINGS

The invention will be described with reference to the accompanying drawings, wherein like numbers reference like elements.

FIG. 1 is a schematic plan sectional view illustrating a structure of an ultrasound emission device according to a first embodiment.

FIG. 2 is a schematic view for describing an installation state of the ultrasound emission device.

FIG. 3 is a schematic sectional view for describing an installation state of the ultrasound emission device.

FIG. 4 is a schematic plan sectional view illustrating a structure of an ultrasound emission device according to a second embodiment.

FIG. 5 is a schematic perspective view for describing an installation state of an ultrasound emission device according to a third embodiment.

FIG. 6 is a schematic plan sectional view illustrating a structure of the ultrasound emission device.

### DESCRIPTION OF EXEMPLARY EMBODIMENTS

Hereinafter, embodiments will be described with reference to the drawings. Each member in each drawing is illustrated with every different scale so that each member has a recognizable size on each drawing.

### First Embodiment

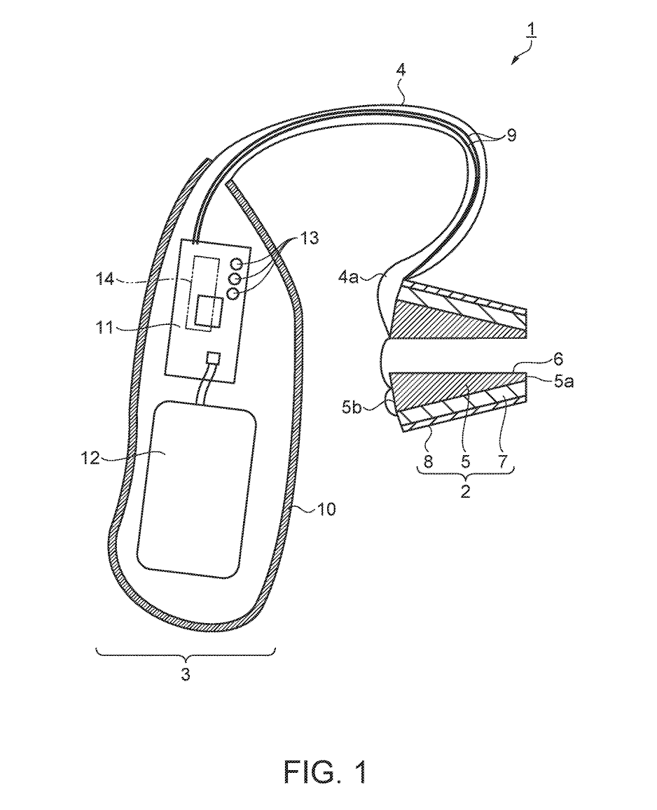

In the present embodiment, a characteristic example of an ultrasound emission device and a method in which an ultrasound gives a stimulus to a brain by using the ultrasound emission device will be described with reference to the drawings. The ultrasound emission device according to a first embodiment will be described with reference to FIGS. 1 to 3. FIG. 1 is a schematic plan sectional view illustrating a structure of the ultrasound emission device. As illustrated in FIG. 1, an ultrasound emission device 1 includes an ultrasound emission unit 2 and a drive unit 3 that drives the ultrasound emission unit 2. The ultrasound emission unit 2 and the drive unit 3 are connected to each other by a support unit 4.

The ultrasound emission unit 2 emits the ultrasound to an examinee. The drive unit 3 drives the ultrasound emission unit 2 by outputting an electrical signal to the ultrasound emission unit 2. The support unit 4 supports the ultrasound emission unit 2 and the drive unit 3 so as to be installed in the examinee. A shape of the support unit 4 is a shape extending along an auditory capsule of the examinee, and is a shape to be installed in the examinee.

The ultrasound emission unit 2 includes a base 5 having a truncated cone shape. The base 5 has a through-hole 6 installed as a hole penetrating an upper surface 5a and a bottom surface 5b. The ultrasound emission unit 2 is installed in the external auditory canal of the examinee. In this case, a sound from an external environment is transmitted to an eardrum through the through-hole 6. An ultrasound array 7 is installed on a side surface of the ultrasound emission unit 2 having a truncated cone shape. In the ultrasound array 7, ultrasound elements are arranged in a matrix form on a substrate. A frequency of the ultrasound emitted by each ultrasound element is not particularly limited. However, for example, the frequency can be selected from a range of 0.25 MHz to 20 MHz. It is preferable to select the frequency depending on the reaction of the examinee. It is preferable that a size of the ultrasound element is a size suitable for the frequency of the ultrasound to be emitted. In this manner, it is possible to efficiently emit the ultrasound.

Although a type of the ultrasound element is not particularly limited, it is possible to use a piezoelectric element such as a lead zirconate titanate (PZT) element and a polyvinylidene fluoride (PDVF) element. The embodiment employs the PZT element which is one of the piezoelectric elements.

A coating film 8 is installed by covering the ultrasound array 7. The coating film 8 may be a waterproof film which mitigates acoustic impedance between the ultrasound array 7 and the skin of the examinee. For example, the embodiment employs a silicone rubber film. It is preferable to coat a gel between the coating film 8 and the skin of the examinee. It is possible to efficiently propagate the ultrasound.

The support unit 4 is connected to the bottom surface 5b of the base 5. The support unit 4 has a tubular shape, and internally has multiple wires 9 installed therein. In the support unit 4, a connection portion 4a connected to the bottom surface 5b is elastic. The connection portion 4a of the support unit 4 is likely to be twisted. In this manner, the support unit 4 is hooked to the auditory capsule, thereby enabling the ultrasound emission unit 2 to be inserted into the external auditory canal.

The drive unit 3 includes a housing 10. A circuit board 11 and a battery 12 are installed inside the housing 10. The housing 10 is openable and closeable, and the battery 12 is installed so as to be detachable from the housing 10. When the capacity of the battery 12 is insufficient, the battery 12 can be replaced. The battery 12 supplies power to the circuit board 11.

In addition to a central processing unit (CPU), the circuit board 11 has a pulse generation circuit, an amplification circuit, a memory, a switch 13, and a display device 14. The wire 9 is connected to the circuit board 11. The pulse generation circuit forms a pulse waveform, and the amplification circuit amplifies the power. The pulse waveform subjected to power amplification is supplied to the ultrasound array 7 via the wire 9. In the ultrasound array 7, the ultrasound element emits the ultrasound by using the pulse waveform.

The examinee operates the switch 13 so as to instruct starting and stopping emission of the ultrasound. Furthermore, a sound pressure level of the ultrasound emitted from the ultrasound emission unit 2 can be adjusted. The CPU detects an input of the switch 13, and outputs an instruction signal for changing a voltage of the pulse waveform to the pulse generation circuit. The pulse generation circuit changes the voltage of the pulse waveform after receiving the instruction signal. In this manner, the sound pressure level of the ultrasound is changed.

The frequency of the ultrasound emitted from the ultrasound emission unit 2 can be adjusted. The CPU detects an input of the switch 13, and outputs an instruction signal for changing the frequency of the pulse waveform to the pulse generation circuit. The pulse generation circuit changes the frequency of the pulse waveform after receiving the instruction signal. In this manner, the frequency of the ultrasound is changed.

Furthermore, the drive unit 3 is provided with a timer function. A time for emitting the ultrasound from the ultrasound emission unit 2 can be adjusted. The CPU detects an input of the switch 13, and stores a drive time in the memory. The ultrasound emission unit 2 starts to emit the ultrasound. When the CPU measures an elapsed time and the elapsed time reaches a preset time, the ultrasound emission unit 2 stops emitting the ultrasound.

The display device 14 displays a sound pressure setting value or the frequency of the ultrasound. In addition, the display device 14 displays a remaining drive time when the timer function is operated.

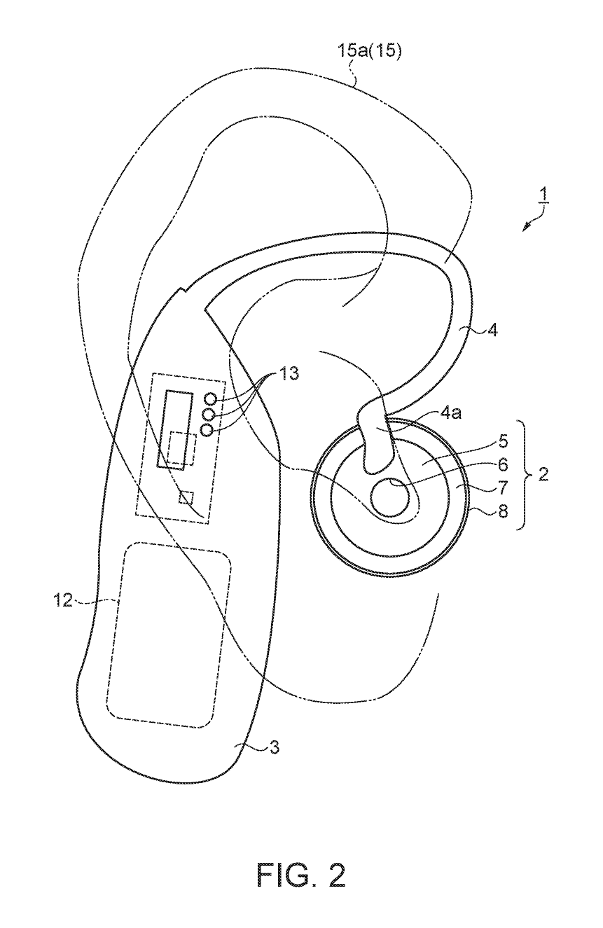

FIG. 2 is a schematic view for describing an installation state of the ultrasound emission device. As illustrated in FIG. 2, the support unit 4 has a shape to be installed in an auditory capsule 15a of an examinee 15. The support unit 4 is disposed along the auditory capsule 15a of the examinee 15 as the human body. The support unit 4 is installed between the head and the auditory capsule 15a. The connection portion 4a is twisted, and the ultrasound emission unit 2 is inserted into an external auditory canal 16. The drive unit 3 is located on a side where gravity acts on the auditory capsule 15a. In the drive unit 3, the battery 12 serves as a center of gravity, and the battery 12 is located on a side where gravity acts on the auditory capsule 15a. In this manner, the ultrasound emission device 1 is stably installed in the auditory capsule 15a.

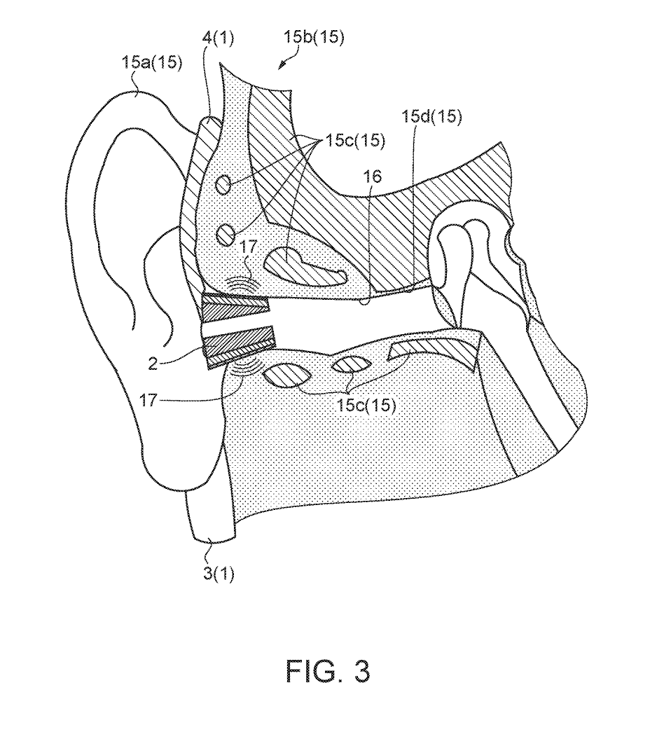

FIG. 3 is a schematic sectional view for describing an installation state of the ultrasound emission device. As illustrated in FIG. 3, the support unit 4 is installed by being hooked to the auditory capsule 15a. The support unit 4 is installed along a head 15b of the examinee 15. In the head 15b, a brain is present inside a skull 15c. The skull 15c has a hole 15d in a location where the external auditory canal 16 is present. An inner surface of the hole 15d is covered with skin tissues.

the ultrasound emission unit 2 has a shape to be installed in the external auditory canal 16 of the examinee 15. The ultrasound emission unit 2 is inserted into the external auditory canal 16. An ultrasound 17 emitted by the ultrasound emission unit 2 is propagated through tissues located in the external auditory canal 16, and reaches the brain. The ultrasound 17 is more likely to be propagated through the tissues located in the external auditory canal 16 than the skull 15c. Even if the ultrasound 17 has small sound pressure, the ultrasound 17 can reach the brain. A microtubule associated with feelings of the examinee 15 is present inside the brain. The microtubule is found in the cell, and has a tubular structure whose diameter is approximately 25 nm. The microtubule present inside the brain which is associated with the feelings is resonated by emitting the ultrasound 17 with the same frequency as that of the ultrasound 17. As a result, symptoms such as depression can be improved.

As described above, according to the embodiment, the following advantageous effects are achieved.

(1) According to the embodiment, the ultrasound emission device 1 includes the ultrasound emission unit 2, the support unit 4, and the drive unit 3. The ultrasound emission unit 2 emits the ultrasound 17. The support unit 4 supports the ultrasound emission unit 2. The drive unit 3 drives the ultrasound emission unit 2. In this manner, the ultrasound emission device 1 can transmit the ultrasound 17 to the brain from the external auditory canal 16 of the examinee 15. The examinee 15 receives the ultrasound 17 from the external auditory canal 16, thereby enabling the brain to be activated and awakened. The support unit 4 installs the ultrasound emission unit 2 and the drive unit 3 in the examinee 15. Therefore, even when the examinee 15 moves, the ultrasound 17 can be emitted toward the examinee 15.

The ultrasound emission unit 2 has a shape to be installed in the external auditory canal 16 of the examinee 15. The support unit 4 has a shape to be installed in the auditory capsule 15a of the examinee 15. Therefore, the ultrasound 17 can be emitted to the external auditory canal 16 by installing the ultrasound emission device 1 in the auditory capsule 15a of the examinee 15. The brain is surrounded with the skull 15c, and the ultrasound 17 is less likely to be transmitted through the skull 15c.

On the other hand, the skull 15c has the hole 15d in the location where the external auditory canal 16 is present. The inner surface of the hole 15d is covered with the skin tissues. The ultrasound emission unit 2 is inserted into the external auditory canal 16. The ultrasound 17 emitted by the ultrasound emission unit 2 is propagated through the tissues located in the external auditory canal 16, and reaches the brain. The ultrasound 17 is more likely to be propagated through the tissues located in the external auditory canal 16 than the skull 15c. Even if the ultrasound 17 has small sound pressure, the ultrasound 17 can reach the brain.

Therefore, the ultrasound emission unit 2 can consume less power. Accordingly, even if the ultrasound emission device 1 employs the small capacity battery 12 which is light in weight, the ultrasound 17 can be emitted to the brain for a long period of time. If the battery 12 is light in weight, the ultrasound emission device 1 also becomes light in weight. If the ultrasound emission device 1 is light in weight, even if the ultrasound emission device 1 is installed in the auditory capsule 15a, the examinee 15 cannot feel a pain. As a result, even when the examinee 15 moves during a daily life, the ultrasound 17 can be transmitted to the brain.

(2) According to the embodiment, the through-hole 6 is installed in the ultrasound emission unit 2. A sound can be transmitted to the eardrum of the examinee 15 through the through-hole 6.

(3) According to the embodiment, the support unit 4 is installed by being hooked to the auditory capsule 15a of the examinee 15. The auditory capsule 15a protrudes from the head 15b. The ultrasound emission device 1 can be mounted thereon, and can be fixed to such an extent that the ultrasound emission device 1 withstands impact made by the movement of the examinee 15.

(4) According to the embodiment, the ultrasound emission unit 2 emits the ultrasound 17 from the external auditory canal 16. In this case, a moderate stimulus can be given to immune system cells of the brain. The immune system cells of the brain reduce plaques. Therefore, it is possible to improve nerve-related diseases inside the brain. In addition, even a person who does not suffer from mental disorders can have improved feelings, when the ultrasound emission device 1 is used so as to give the stimulus to the brain by emitting the ultrasound 17.

### Second Embodiment

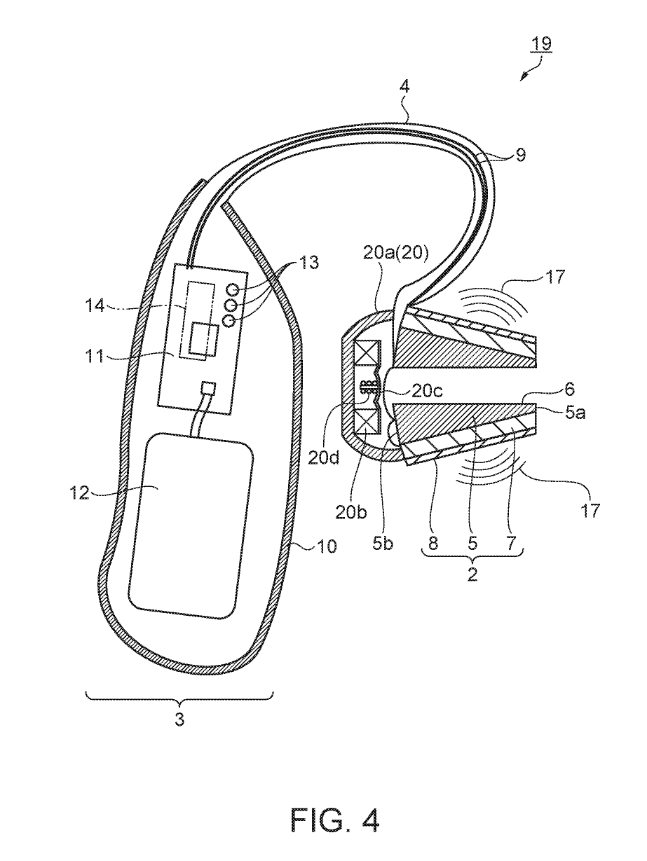

Next, an embodiment of the ultrasound emission device will be described with reference to a schematic plan sectional view illustrating a structure of the ultrasound emission device in FIG. 4. The embodiment is different from the first embodiment in that a speaker for emitting a voice is installed therein. With regard to points which are the same as those in the first embodiment, description will be omitted.

That is, according to the embodiment, as illustrated in FIG. 4, a speaker 20 is installed on the bottom surface 5b of the ultrasound emission unit 2 in the ultrasound emission device 19. The speaker 20 includes a case 20a having a substantially bottomed cylindrical shape. A cylindrical permanent magnet 20b is installed on a bottom surface of the case 20a. A diaphragm 20c is installed on a surface of the permanent magnet 20b on the ultrasound emission unit 2 side. A voice coil 20d is installed on a surface of the diaphragm 20c on the permanent magnet 20b side. A current signal having a voice waveform is applied to the voice coil 20d, thereby vibrating the diaphragm 20c. A voice is generated from the diaphragm 20c. The voice reaches the eardrum of the examinee 15 through the through-hole 6, thereby vibrating the eardrum.

The circuit board 11 is connected to the voice coil 20d by the wire 9. The memory installed in the circuit board 11 stores voice data. A digital to analog (D/A) converter is installed in the circuit board 11. The CPU installed in the circuit board 11 outputs the voice data to the D/A converter. The D/A converter converts the voice data into a voice signal, and outputs the voice signal to the voice coil 20d. In this manner, the ultrasound emission device 19 can reproduce the voice data.

As described above, according to the embodiment, the following advantageous effects are achieved.

(1) According to the embodiment, the ultrasound emission device 19 includes the speaker 20 that emits the voice. Therefore, a stimulus using the ultrasound 17 and a stimulus using the voice can be simultaneously given to the examinee 15.

### Third Embodiment

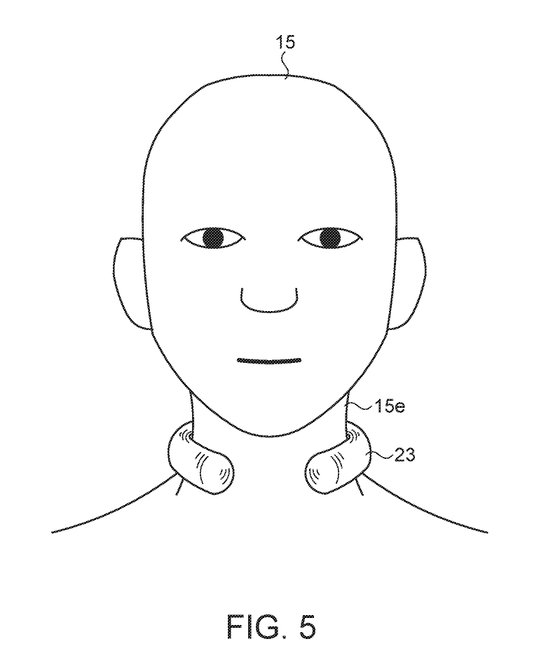

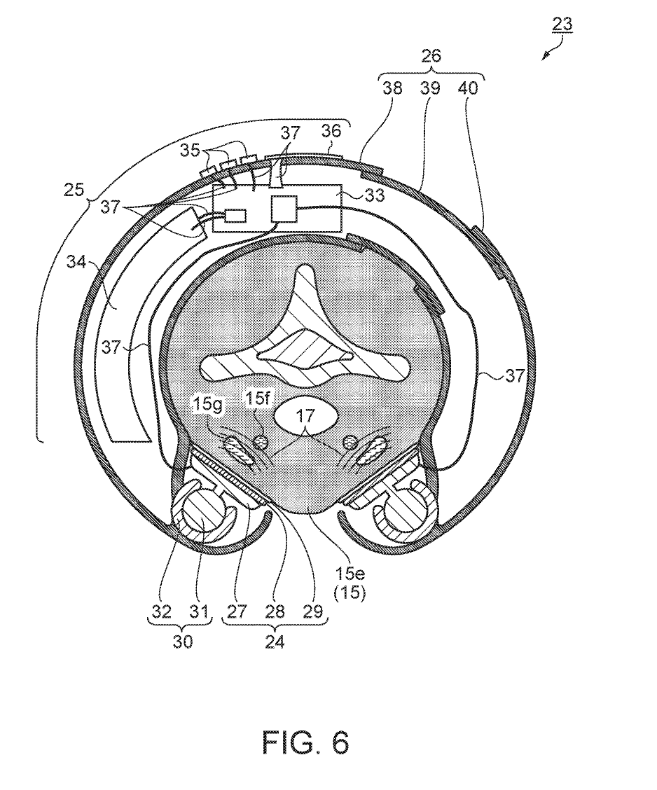

Next, an embodiment of an ultrasound emission device will be described with reference to FIGS. 5 and 6. The embodiment is different from the first embodiment in that the ultrasound emission device is installed on a neck 15e of the examinee 15. With regard to points which are the same as those in the first embodiment, description will be omitted.

FIG. 5 is a schematic perspective view for describing an installation state of the ultrasound emission device. That is, according to the embodiment, as illustrated in FIG. 5, an ultrasound emission device 23 is used by being installed on the neck 15e of the examinee 15. The ultrasound emission device 23 has an annular shape which is partially cut out, and is wound along an outer periphery of the neck 15e.

FIG. 6 is a schematic plan sectional view illustrating a structure of the ultrasound emission device. As illustrated in FIG. 6, the ultrasound emission device 23 includes an ultrasound emission unit 24 and a drive unit 25 that drives the ultrasound emission unit 24. The ultrasound emission unit 24 and the drive unit 25 are supported by a support unit 26.

The ultrasound emission unit 24 emits the ultrasound 17 toward the neck 15e of the examinee 15. The drive unit 25 outputs an electrical signal to the ultrasound emission unit 24 so as to drive the ultrasound emission unit 24. The support unit 26 supports the ultrasound emission unit 24 and the drive unit 25 so as to be installed on the neck 15e of the examinee 15. A shape of the support unit 26 is a shape extending along the neck 15e of the examinee 15, and is a shape to be installed on the neck 15e.

The ultrasound emission unit 24 includes a disc-shaped base 27. An ultrasound array 28 is installed in the base 27 on the neck 15e side. The ultrasound array 28 is provided with the same structure and the same function as the ultrasound array 7 according to the first embodiment. A coating film 29 is installed by covering the ultrasound array 28. The coating film 29 may be a waterproof film which mitigates acoustic impedance between the ultrasound array 28 and the skin of the examinee 15. For example, the embodiment employs a silicone rubber film. It is preferable to coat a gel between the coating film 29 and the skin of the examinee 15. It is possible to efficiently propagate the ultrasound 17.

The ultrasound emission unit 24 is connected to an emission direction adjustment unit 30. The emission direction adjustment unit 30 has a spherical bearing structure in which a spherical portion 31 is accommodated inside a housing 32. The base 27 is connected to the spherical portion 31. The emission direction adjustment unit 30 can change a direction of a surface from which the ultrasound 17 is emitted by the ultrasound emission unit 24. When the coating film 29 of the ultrasound emission unit 24 is brought into contact with the skin of the neck 15e, the coating film 29 is disposed along the surface of the neck 15e. Therefore, the surface from which the ultrasound 17 is emitted can match the surface of the neck 15e. As a result, the ultrasound 17 can be efficiently emitted to the inside of the neck 15e.

An artery 15f and a vein 15g are present inside the neck 15e. The ultrasound emission unit 24 is installed at a location facing the artery 15f and the vein 15g. When the ultrasound emission unit 24 emits the ultrasound 17, the ultrasound 17 can be transmitted to the artery 15f and the vein 15g. In this manner, the artery 15f and the vein 15g can be vibrated so as to stimulate an organ inside the brain.

The inside of the support unit 26 has a cylindrical shape, and the inside of the support unit 26 is hollow. A circuit board 33 and a battery 34 are installed inside the support unit 26. The support unit 26 is partially openable and closeable. The battery 34 is installed so as to be detachable from the support unit 26. When the capacity of the battery 34 is insufficient, the battery 34 can be replaced. The battery 34 supplies power to the circuit board 33.

In addition to the CPU, the circuit board 33 has a pulse generation circuit, an amplification circuit, and a memory. A switch 35 and a display device 36 are installed on a surface of the support unit 26. The switch 35 and the display device 36 are connected to the circuit board 33 by a wire 37. Furthermore, the ultrasound array 28 is connected to the circuit board 33 by the wire 37. The pulse generation circuit forms a pulse waveform, and the amplification circuit amplifies the power. The pulse waveform subjected to power amplification is supplied to the ultrasound array 28 via the wire 37. In the ultrasound array 28, the ultrasound element emits the ultrasound by using the pulse waveform. In this way, the drive unit 25 is provided with the same configuration and the same function as the drive unit 3 according to the first embodiment. The examinee 15 can start and stop emission of the ultrasound by operating the switch 35, and can change the sound pressure level of the ultrasound, the frequency of the ultrasound, and the drive time.

The support unit 26 includes a first housing 38, and a second housing 39 and a third housing 40 which serve as support adjustment units. An end of the second housing 39 is inserted into the first housing 38, and the other end of the second housing 39 is inserted into the third housing 40. Therefore, a portion of the second housing 39 overlaps the first housing 38, and a portion of the second housing 39 overlaps the third housing 40.

The examinee 15 can change a length in which the second housing 39 overlaps the first housing 38 and the third housing 40 by operating the support unit 26. The examinee 15 changes an inner diameter of the support unit 26. In this manner, the examinee 15 can adjust a shape of the support unit 26 so as to match a cross-sectional shape of the neck 15e of the examinee 15. Therefore, even when the examinee 15 moves, the ultrasound emission device 23 can stably emit the ultrasound 17 to the artery 15f and the vein 15g.

As described above, according to the embodiment, the following advantageous effects are achieved.

(1) According to the embodiment, the ultrasound emission device 23 is installed on the neck 15e of the examinee 15 by using the support unit 26. The ultrasound emission unit 24 emits the ultrasound 17 toward the neck 15e of the examinee 15. Therefore, the ultrasound 17 can be emitted to the neck 15e by installing the ultrasound emission device 23 on the neck 15e of the examinee 15. As a result, the ultrasound emission device 23 can give a stimulus of the ultrasound 17 to the brain by way of the artery 15f and the vein 15g of the neck 15e.

The ultrasound emission unit 24 has a shape to be installed on the neck 15e of the examinee 15. The support unit 26 has a shape to be installed on the neck 15e of the examinee 15. Therefore, the ultrasound 17 can be emitted to the artery 15f and the vein 15g of the neck 15e by installing the ultrasound emission device 23 on the neck 15e of the examinee 15. The brain is surrounded with the skull 15c, and the ultrasound 17 is less likely to be transmitted through the skull 15c. On the other hand, since the neck 15e has no skull 15c, even low sound pressure enables the ultrasound 17 to be propagated to the artery 15f and the vein 15g. A stimulus of the ultrasound 17 can be propagated to the brain via the artery 15f and the vein 15g. Therefore, even if the ultrasound emission device 23 employs the small capacity battery 34 which is light in weight, the ultrasound 17 can be emitted to the artery 15f and the vein 15g for a long period of time. If the ultrasound emission device 23 is light in weight, even if the ultrasound emission device 23 is installed on the neck 15e, the examinee 15 cannot feel a pain. As a result, even when the examinee 15 moves during a daily life, the stimulus can be transmitted to the brain by emitting the ultrasound 17.

(2) According to the embodiment, the ultrasound emission unit 24 has the emission direction adjustment unit 30 that changes a direction of the surface from which the ultrasound 17 is emitted by the ultrasound emission unit 24. Therefore, the surface from which the ultrasound 17 is emitted can match the surface of the neck 15e. As a result, the ultrasound 17 can be efficiently transmitted to the artery 15f and the vein 15g.

(3) According to the embodiment, the support unit 26 has the second housing 39 whose shape is deformed so as to match the neck 15e of the examinee 15. Therefore, even when the examinee 15 moves, the ultrasound emission device 23 can stably emit the ultrasound 17 to the artery 15f and the vein 15g.

(4) According to the embodiment, the support unit 26 is installed on the neck 15e of the examinee 15. The neck 15e protrudes in a columnar shape from the body. The ultrasound emission device 23 can be mounted thereon, and can be fixed to such an extent that the ultrasound emission device 23 withstands impact made by the movement of the examinee 15. As a result, the examinee 15 is active during a daily life, a stimulus can be given to the brain by emitting the ultrasound 17 via the artery 15f and the vein 15g.

The invention is not limited to the above-described embodiments. Various modification or improvements can be added to the invention within the technical spirit of the invention by those who have a normal knowledge in the art. Modification examples will be described below.

### Modification Example 1

According to the above-described first embodiment, the ultrasound emission device 1 is fixed to the auditory capsule 15a by the support unit 4. Alternatively, like a headphone, the support unit 4 may be installed at the top of the head of the examinee 15. The ultrasound emission unit 2 may be installed in the right and left external auditory canals 16. A stimulus of the ultrasound 17 can be given to the brain from the right and left external auditory canals 16.

### Modification Example 2

According to the above-described third embodiment, the ultrasound emission device 23 emits the ultrasound 17 to the neck 15e. An earphone may be installed in the ultrasound emission device 23. The ultrasound 17 is emitted to the artery 15f and the vein 15g, and a voice may be heard through the ear. Both stimuli of the ultrasound 17 and the voice can be given to the brain.

The entire disclosure of Japanese Patent Application NO. 2016-038647 filed on Mar. 1, 2016 is expressly incorporated by reference herein.

What is claimed is:

1. An ultrasound emission device comprising:
an ultrasound emission unit that emits an ultrasound;
a drive unit that drives the ultrasound emission unit; and
a support unit that supports the ultrasound emission unit and the drive unit so as to be installed in a human body,
wherein the ultrasound emission unit has a shape to be installed in an external auditory canal of the human body, and
wherein the support unit has a shape to be installed in an auditory capsule of the human body.

2. The ultrasound emission device according to claim 1,
wherein the ultrasound emission unit has a hole installed in order to transmit a sound to an eardrum of the human body.

3. The ultrasound emission device according to claim 2, further comprising:
a speaker that emits a voice.

4. An ultrasound emission device comprising:
an ultrasound emission unit that emits an ultrasound;
a drive unit that drives the ultrasound emission unit; and
a support unit that supports the ultrasound emission unit and the drive unit so as to be installed in a human body,
wherein the support unit has a shape to be installed on a neck of the human body, and
wherein the ultrasound emission unit emits the ultrasound toward the neck of the human body.

5. The ultrasound emission device according to claim 4,
wherein the ultrasound emission unit has an emission direction adjustment unit that changes a direction of a surface from which the ultrasound is emitted.

6. The ultrasound emission device according to claim 4,
wherein the support unit has a support adjustment unit that is deformed so as to match the neck of the human body.
